(19) United States
(12) Patent Application Publication
(10) Pub. No.: US 2021/0093729 A1
(43) Pub. Date: Apr. 1, 2021

(54) PREPARATION AND APPLICATION OF SURFACE DOUBLE MODIFIED HUMAN SERUM ALBUMIN AS TARGETING NANO DRUG CARRIER

(71) Applicant: TIANJIN UNIVERSITY OFR SCIENCE AND TECHNOLOGY, Tianjin (CN)

(72) Inventors: Na GUO, Tianjin (CN); Peng YU, Tianjin (CN); Fengyan LI, Tianjin (CN); Jingjing TANG, Tianjin (TW); Tiantian HAO, Tianjin (CN); Ying FU, Tianjin (CN); Haomeng WANG, Tianjin (CN); Yuou TENG, Tianjin (CN); Dong ` WANG, Tianjin (CN)

(21) Appl. No.: 17/032,540

(22) Filed: Sep. 25, 2020

Related U.S. Application Data
(60) Provisional application No. 62/906,286, filed on Sep. 26, 2019.

(51) Int. Cl. A61K 47/64; A61K 9/14; A61K 31/337

(52) U.S. Cl. CPC A61K 47/643; A61K 31/337; A61K 9/146

(57) ABSTRACT

Disclosed herein is a preparation method and use of a surface double-modified human serum albumin as a tumor targeting nano drug carrier, wherein the targeting nano drug carrier includes human serum albumin (HSA), polyethylene glycol and E-selectin peptide ligand. The targeting nano drug carrier is formed by coupling the E-selectin peptide ligand and polyethylene glycol to the surface of HSA by thioether linker formed between the sulfhydryl group of E-selectin peptide ligand and activated HSA. The targeting nano drug carrier can encapsulate hydrophobic antitumor drugs and form nanoparticles with certain particle size after treatment, which can not only increase the solubility and stability of the hydrophobic antitumor drugs, but also can actively recognize and bind E-selectin which is highly expressed on activated endothelial cells at tumor site, competitively inhibit the interaction between E-selectin and tumor cells, and thus inhibit the adhesion and migration of tumor cells.

This application claims the benefit of U.S. Provisional Application No. 62/906,286, filed Sep. 26, 2019, the content of which is incorporated herein by reference in its entirety.

This disclosure generally relates to the field of nano drug technology, more specifically to a targeting nano drug carrier, a preparation method and application thereof.

### BACKGROUND

HSA is the most abundant protein in plasma. It has a long half-life in vivo. Its three-dimensional structure is heart-shaped and contains multiple ligand binding sites. It is a natural carrier for the transport of hydrophobic substances in vivo. It can transport various substances to the corresponding tissues through reversible binding. The properties of HSA are relatively stable, and it has a certain degree of tolerance to acid-base environment, temperature and organic solvents, so it is suitable for the preparation of a variety of nanoparticles. There are many carboxyl and amino groups on the surface of human serum albumin, which can be modified by covalent bond, surface coating or electrostatic adsorption. The modified nano drug carrier of serum albumin not only has outstanding drug loading capacity, but also can optimize the pharmacokinetic parameters of drugs. Abraxane, approved by FDA in 2005, is the HSA binding form of paclitaxel, and the toxicity is greatly reduced compared with paclitaxel.

E-selectin is associated with many diseases and is highly expressed only in activated endothelial cells. E-selectin can specifically recognize the terminal domains of some glycoproteins and glycolipid molecules on the surface of tumor cells. Through this interaction between E-selectin and tumor cells, tumor cells can adhere to endothelial cells, and then migrate with blood flow, resulting in the migration of tumor cells. Therefore, E-selectin can be used as a therapeutic intervention target.

The structural characteristics of natural ligands for E-selectin have not been fully elucidated. Some studies have suggested that the natural ligands specifically recognized by E-selectin may include sugars, peptides and other structural types. At present, the most widely accepted natural ligand structure of E-selectin is Sialyl Lewis X (sLex). The specific ligand of E-selectin can be used as a target molecule to bring other anti-tumor drugs to the tumor site, and it can also inhibit the migration of tumor cells. Therefore, the conjugates of E-selectin ligand and anti-tumor drugs are expected to achieve dual therapeutic effect and reduced toxicity.

Polyethylene glycol has high hydrophilicity, large hydrodynamic volume in aqueous solution and no immunogenicity. When coupled to drug molecules or drug surface, the modified drug molecules can be endowed with excellent properties, their biodistribution behavior and solubility in aqueous solution can be improved, and the drug circulation time in vivo can be prolonged.

Nano carriers have been widely used in the delivery of anticancer drugs to improve the solubility of drugs in water and improve the passive targeting of tumors through EPR effect, improving the therapeutic effect. However, there is still a lack of active targeting capacity for HSA based nanodrugs. More importantly, there is no HAS-based drug carrier that can not only target and deliver drugs, but also inhibit tumor metastasis.

### SUMMARY

This disclosure provides a preparation method and use of E-selectin peptide ligand modified human serum albumin targeting nano drug carrier. The targeting nano drug carrier comprises human serum albumin (HSA), polyethylene glycol and E-selectin peptide ligand. The targeting nano drug carrier is formed by coupling the E-selectin peptide ligand and polyethylene glycol to the surface of HSA with a thioether linker which is formed by the sulfhydryl group of peptide ligand and activated HSA. The targeting nano drug carrier can encapsulate hydrophobic antitumor drugs and form nanoparticles with certain particle size, which can not only increase the solubility and stability of hydrophobic anti-tumor drugs, but also can recognize and bind E-selectin which is highly expressed on activated endothelial cells, competitively inhibit the binding of tumor cells and E-selectin to inhibit tumor cell adhesion and migration.

The objectives of the invention are realized through the following embodiments.

In some embodiments, the present disclosure provides a HSA-based targeting nano drug carrier comprising human serum albumin (HSA), polyethylene glycol (PEG) and E-selectin peptide ligand, PEG and E-selectin peptide ligand are coupled to the surface of HSA.

In some embodiments, the sequence of E-selectin peptide ligand is CIELLQAR, its reverse sequence analog thereof, or a peptide analog comprising an amino acid in a non-naturally occurring configuration (D-configuration).

In some embodiments, one or more of hydrophobic antitumor drugs can be loaded on targeting nano drug carriers, wherein the hydrophobic antitumor drugs can include paclitaxel, doxorubicin, docetaxel etc.

In some embodiments, the molecular weight of the polyethylene glycol is from 1500 to 20000.

In some embodiments, the present disclosure provides a synthetic method of surface double modified human serum albumin as targeting nano drug carrier, characterized in comprising the following steps:

(1) synthesizing a conjugate of E-selectin ligand and mPEG by Fmoc solid phase synthesis.

(2) coupling the conjugate of E-selectin ligand and mPEG with activated human serum albumin to obtain HSA monomer carrier.

In some embodiments, step (1) is as follows:

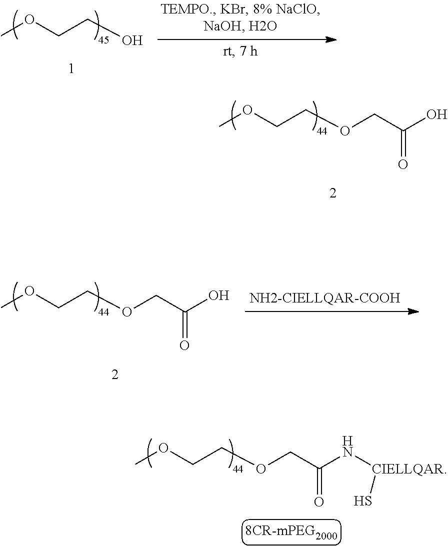

In some embodiments, step (2) is as follows:

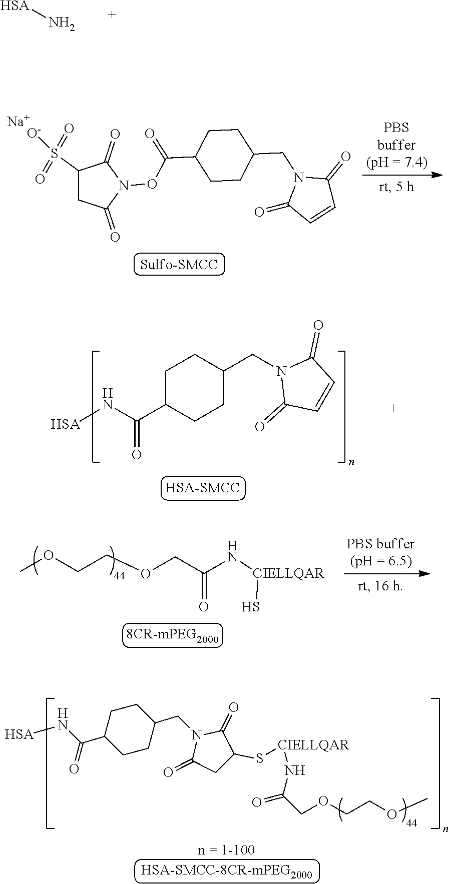

In some embodiments, the modified HSA monomer can be dissolved in a buffer solution, β-mercaptoethanol can be added to break the intermolecular disulfide bond, the drug to be loaded can be added, and the free drug can be removed by ultrafiltration and centrifugation, and the drug loaded nanoparticles can be obtained by freeze-drying.

Also provided is a use of the targeting nano drug carrier in the delivery of hydrophobic antitumor drugs.

### Alternative Exemplary Embodiments 1-27

Embodiment 1. A conjugate comprising a) albumin unit, b) polyethylene glycol unit, and c) an E-selectin peptide ligand unit, wherein the polyethylene glycol and the E-selectin peptide ligand are conjugated to the albumin. Although albumin is preferred protein for preparing the conjugates of embodiments herein, the present disclosure is not limited to only albumin, other proteins e.g., antibodies, can also be used for preparing the conjugates in replace of albumin in certain embodiments.

Embodiment 2. The conjugate of embodiment 1, wherein the albumin is human serum albumin

Embodiment 3. The conjugate of embodiment 1 or 2, wherein the polyethylene glycol and the E-selectin peptide ligand are conjugated to the surface of the albumin.

Embodiment 4. The conjugate of any one of embodiments 1-3, wherein the E-selectin peptide ligand is a polypeptide having a sequence of CIELLQAR, a reversed sequence analog thereof, (i.e., with sequence of RAQLLEIC), or a peptide analog thereof wherein one or more of the amino acids are in a non-naturally occurring configuration (D-configuration).

Embodiment 5. The conjugate of any one of embodiments 1-4, wherein the polyethylene glycol is characterized as having an average number molecular weight (Mn) of about 1500 to 20,000, for example, 2000, 3000, 5000, 10000, 20000, or any ranges between the recited values.

Embodiment 6. The conjugate of any one of embodiments 1-5, which is characterized by the general formula I:

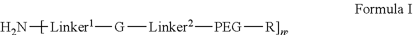

wherein:
HSA represents human serum albumin,
Linker¹ is a linker that connects G with HSA, e.g., described herein,
G is the E-selectin peptide ligand unit, e.g., having the sequence of CIELLQAR, preferably, G connects to Linker¹ and Linker² through the same amino acid unit, e.g., through the —SH and —NH2 of the cysteine unit,
Linker² is a linker (e.g., an amide bond, described herein,) that connects G with PEG, which is a polyethylene glycol unit,
R is a capping group of the polyethylene glycol unit, such as hydrogen or Methyl group,
n is an integer ranging from 1-100 (e.g., 1, 2, 3, 4, 5, 6, 7, 8, 9, 10, 11, 12, 13, 14, 15, 20, 25, 30, 40, or 50, or any ranges between the recited values). It is to be appreciated that Linker' typically is a bifunctional molecule, with one end capable of forming a covalent bond with a protein, such as HSA, which can be through forming an amide bond with the protein, and the other end can form a covalent bond with G, for example, through a 1,4-addition reaction between an alpha-beta-unsaturated carbonyl compound and a thiol group. Any of such linkers known in the art, for example, those used for an antibody-drug-conjugates, can be used for embodiments herein. The number of units that are conjugated to HSA can vary, for example, the number “n” can be 1, 2, 3, 4, 5, etc.

Embodiment 7. The conjugate of embodiment 6, which is characterized by the following formula I-1:

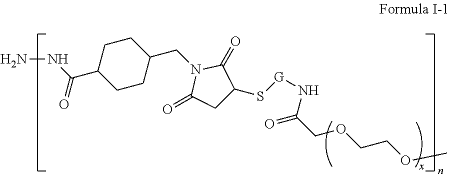

wherein G is the E-selectin peptide ligand having the sequence of CIELLQAR, wherein G connects with the succinimide unit through the cysteine —SH group, and G connects with the PEG unit through the cysteine —NH₂ group,
wherein x is an integer ranging from 1-500 (e.g., 1, 5, 10, 20, 25, 30, 35, 40, 45, 50, 60, 70, 80, 90, 100, 200, 300, 400, 500, or any ranges between the recited values).

Embodiment 8. The conjugate of embodiment 7, wherein x is 44.

Embodiment 9. A method of producing a conjugate comprising:
a) reacting albumin, preferably human serum albumin, with a linker molecule to provide an activated albumin, wherein the activated albumin is capable of reacting with a —SH group to form a covalent bond, and
b) reacting the activated albumin with an E-selectin peptide ligand conjugated with a polyethylene glycol unit, through a —SH group of a cysteine unit of the E-selectin peptide ligand to form the conjugate.

Embodiment 10. The method of embodiment 9, wherein the E-selectin peptide ligand is conjugated with a polyethylene glycol unit having an average number molecular weight (Mn) of about 1500 to 20,000, for example, 2000, 3000, 5000, 10000, 20000, or any ranges between the recited values.

Embodiment 11. The method of embodiment 9 or 10, wherein reacting albumin with a linker molecule comprises reacting human serum albumin with Sulfo-SMCC,

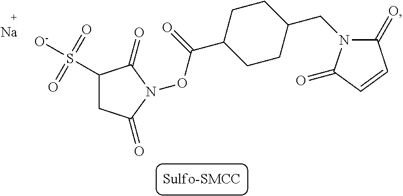

in a buffer, such as a PBS buffer with pH of 7.8, to form the activated human serum albumin, HSA-SMCC,

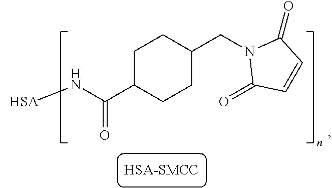

wherein HSA represents human serum albumin,
wherein n is an integer ranging from 1-100 (e.g., 1, 2, 3, 4, 5, 6, 7, 8, 9, 10, 11, 12, 13, 14, 15, 20, 25, 30, 40, or 50, or any ranges between the recited values).

Embodiment 12. The method of any one of embodiments 9-11, wherein reacting the activated albumin with an E-selectin peptide ligand conjugated with a PEG unit comprises reacting HSA-SMCC with

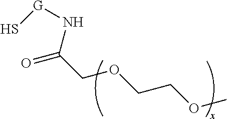

through the SH group to form the conjugate,
wherein G is the E-selectin peptide ligand, preferably, having the sequence of CIELLQAR, wherein the —SH group and —NH group are both derived from the cysteine unit of G,
wherein x is an integer ranging from 1-500 (e.g., 1, 5, 10, 20, 25, 30, 35, 40, 45, 50, 60, 70, 80, 90, 100, 200, 300, 400, 500, or any ranges between the recited values).

Embodiment 13. The method of embodiment 12, wherein x is 44.

Embodiment 14. The method of any one of embodiments 9-13, further comprising purifying HSA-SMCC through dialysis with a molecular weight cut off of 8000 to 14000, e.g., prior to reacting with the E-selectin peptide ligand conjugated with a PEG unit. In some embodiments, dialysis with other molecular weight cut off numbers can also be used for the purification herein.

Embodiment 15. The method of any one of embodiments 9-14, further comprising purifying the conjugate through dialysis with a molecular weight cut off of 3500. In some embodiments, dialysis with other molecular weight cut off numbers can also be used for the purification herein.

Embodiment 16. The conjugate prepared by any one of the methods of embodiments 9-15. In some embodiments, the present disclosure is also directed to the specific conjugates prepared by the methods described herein, such as shown in the Examples section.

Embodiment 17. A pharmaceutical carrier comprising the conjugate of any one of embodiments 1-8 and 16 as a powder or in an aqueous composition.

Embodiment 18. The pharmaceutical carrier of embodiment 17, characterized as having an average particle size diameter of less than 200 nm (e.g., less than 180 nm, less than 160 nm, less than 140 nm, less than 100 nm, etc. or ranging from 100 nm to 200 nm, e.g., ranges between any of 100 nm, 120 nm, 140 nm, 160 nm, 180 nm, and 200 nm, such as 100 nm to 180 nm, 100 nm to 160 nm, etc.).

Embodiment 19. A pharmaceutical composition comprising the conjugate of any one of embodiments 1-8 and 16 or the pharmaceutical carrier of embodiments 17 or 18, and an anticancer drug, preferably, a water insoluble anticancer drug, or a hydrophobic anticancer drug, which is bound to the conjugate. Any of the known anticancer drugs, particularly those water insoluble anticancer drugs, such as those having a water solubility at a pH of about 7 less than 5 mg/mL, less than 1 mg/mL, or less than 0.1 mg/mL, can be used for the embodiments herein. The water insoluble anticancer drugs can typically be hydrophobic anticancer drugs.

Embodiment 20. The pharmaceutical composition of embodiment 19, characterized as having an average particle size diameter of less than 200 nm (e.g., less than 180 nm, less than 160 nm, less than 140 nm, less than 100 nm, etc. or ranging from 100 nm to 200 nm, e.g., ranges between any of 100 nm, 120 nm, 140 nm, 160 nm, 180 nm, and 200 nm, such as 100 nm to 180 nm, 100 nm to 160 nm, etc.). For example, in some embodiments, the pharmaceutical composition can have particles formed from the anticancer drug and the conjugate with an average particle size diameter of about 100 nm to about 200 nm.

Embodiment 21. The pharmaceutical composition of embodiment 19 or 20, wherein the anticancer drug is a taxane, such as paclitaxel or docetaxel, or adriamycine.

Embodiment 22. The pharmaceutical composition of any one of embodiments 19-21, wherein the weight ratio of the conjugate to the anticancer drug is from about 1:1 to about 20:1 (e.g., about 2:1, 3:1, 4:1, 5:1, 6:1, 7:1, 8:1, 9:1, 10:1, 15:1, 20:1 or any ranges between the recited values).

Embodiment 23. A method of preparing any one of the pharmaceutical composition of embodiments 19-22, the method comprising mixing the conjugate in an aqueous solution, such as water or a buffer solution, with an agent, preferably, β-mercaptoethanol, in an effective amount to reduce intramolecular disulfide bonds to form a reduced conjugate; mixing the reduced conjugate with the anticancer drug; oxidizing the reduced conjugate with an added oxidant, e.g., hydrogen peroxide, or with a natural oxidant, e.g., oxygen in the air, reform intramolecular disulfide bonds; removing the anticancer drug that is not bound to the conjugate, e.g., through centrifugal ultrafiltration; and optionally lyophilizing the solution to form a powder. In some embodiments, the present disclosure is also directed to the specific pharmaceutical compositions prepared by the methods described herein, such as shown in the Examples section.

Embodiment 24. A method of treating cancer (e.g., leukemia, pancreatic cancer, lung cancer, breast cancer, or any of the cancer indicated for Abraxane or those described in U.S. Pat. Nos. 7,820,788, 7,758,891, 7,923,536, 8,034,375, 8,138,229, 8,268,348, 8,314,156, 8,853,260, 9,101,543, 9,393,318, 9,511,046, and 9,597,409, the content of each of which is herein incorporated by reference in its entirety), comprising administering to a subject in need thereof a therapeutically effective amount of any one of the pharmaceutical composition of embodiments 19-22, preferably, the method provides less side effect compared to administering a dose equivalent anticancer drug without the conjugate.

Embodiment 25. A method of treating cancer metastasis comprising administering to a subject in need thereof a therapeutically effective amount of any one of the pharmaceutical composition of embodiments 19-22.

Embodiment 26. A pharmaceutical composition comprising the conjugate of any one of embodiments 1-8 and 16 or the pharmaceutical carrier of embodiments 17 or 18, and a drug, preferably, a water insoluble drug (see e.g., U.S. Pat. No. 7,820,788, 7,758,891, 7,923,536, 8,034,375, 8,138,229, 8,268,348, 8,314,156, 8,853,260, 9,101,543, 9,393,318, 9,511,046, and 9,597,409), which is bound to the conjugate.

Embodiment 27. Any and all the embodiments described in CN108578369, the content of which is hereby incorporated by reference in its entirety.

The advantages and potential effects of the present invention include the following:

1. PEG and peptide ligand doubly modified human serum albumin has not been reported.

2. The modified HAS-based carrier has a particle size (100-200 nm) suitable for exerting EPR effect, and the E-selectin peptide can not only be used as a target molecule to bring other anti-tumor drugs to the tumor site, but also can inhibit the migration of tumor cells, so as to achieve the goal of dual therapeutic effect and toxicity reduction.

3. The PEG modified human serum albumin as drug carrier can increase the water solubility and stability of the delivery sytstem, improve the half-life of the plasma, enhance the bioavailability, reduce the immunogenicity of the protein, and improve the drug efficacy and safety.

### BRIEF DESCRIPTION OF THE DRAWINGS

FIG. 1 shows the Western diagram of modified HSA monomer as targeting nano drug carrier in Examples 3 and 4 of this application.

FIG. 2 shows the anti-adhesion activity of the targeting nano drug carrier against the interaction between HUVEC and HL-60 cells according to Example 6 of this application.

FIG. 3 shows the particle size distribution (DLS) of the drug loaded nanoparticles in Example 5 of this application.

FIG. 4 shows zeta potential of the drug loaded nanoparticles in Example 5 of this application.

FIG. 5 shows the change of particle size of the drug loaded nanoparticles at different time in Example 5 of this application.

FIG. 6 shows the atomic force microscope diagram of the drug loaded nanoparticles in Example 5 of this application.

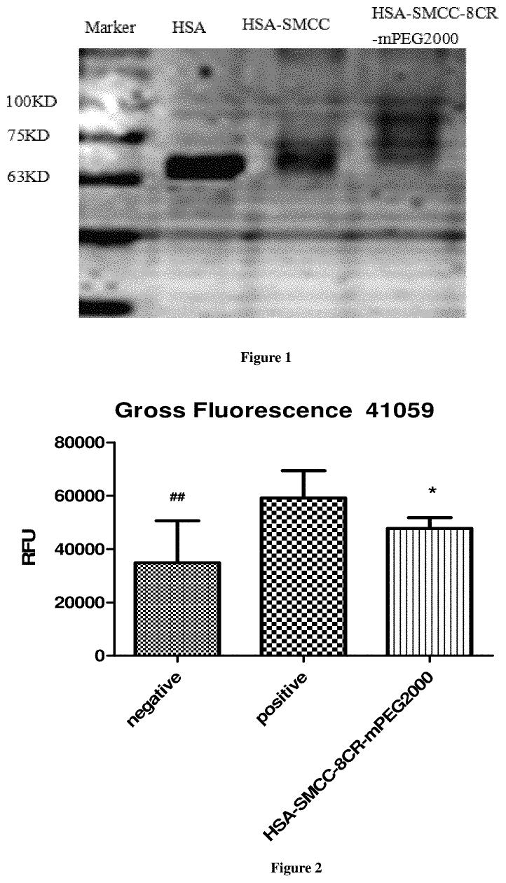

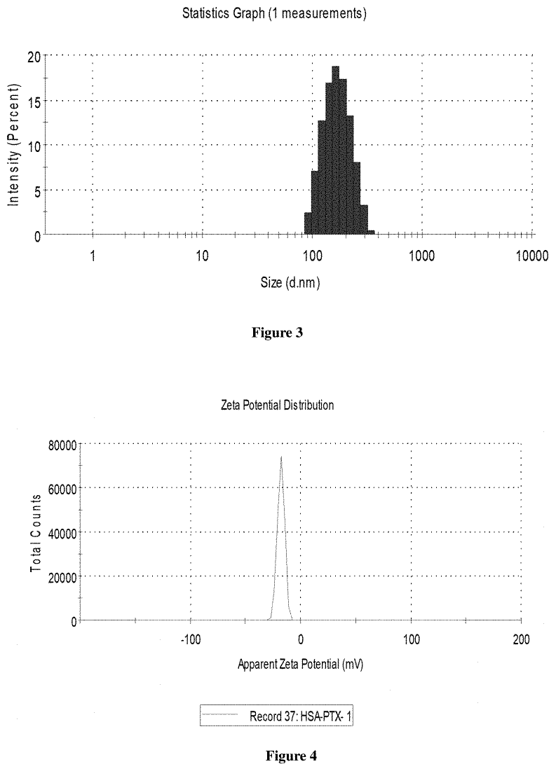

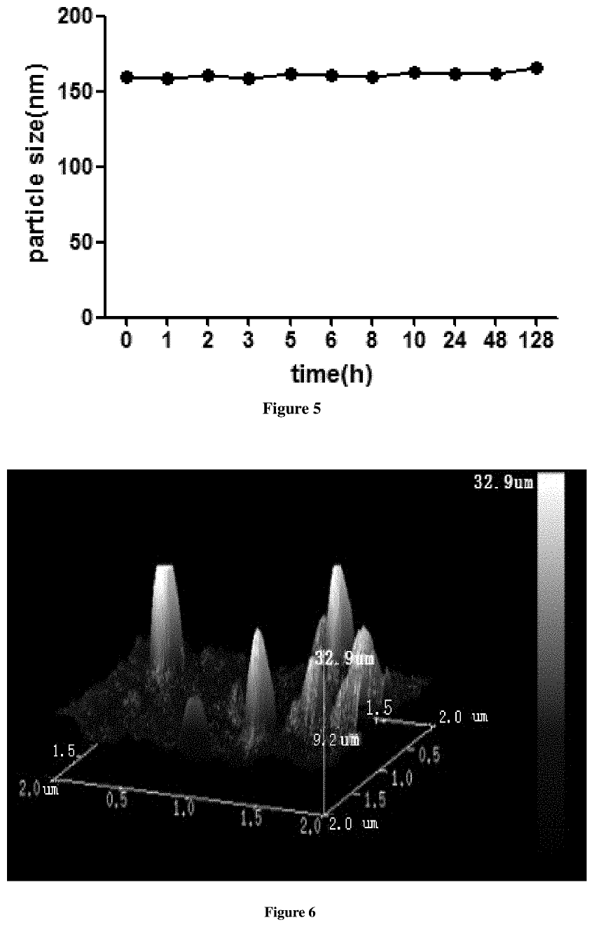

FIGS. 1-6 are representative spectrum obtained for the respective examples and are not to be construed as limiting in any way.

### DETAILED DESCRIPTION

In order to understand the present invention, the following further describes the present invention in connection with the Examples. The following examples are illustrative but not restrictive, and the scope of the present invention is not to be limited by the following examples.

The purpose of the present invention includes modifying the surface of human serum albumin with E-selectin peptide ligand and polyethylene glycol, providing a targeting drug carrier. This targeting drug carrier can load hydrophobic antitumor drugs and form nanoparticles, so as to passively target on tumor site through EPR effect, and at the same time, use E-selectin ligand to actively target angiogenesis, because E-selectin is highly expressed in tumor neovascularization endothelial cells. Moreover, E-selectin ligand can inhibit tumor metastasis by competing with tumor cells to bind E-selectin.

The sequence of E-selectin peptide ligands is CIELLQAR (8CR), its reverse sequence analog thereof, or a peptide analog comprising an amino acid in a non-naturally occurring configuration (D-configuration). These peptides can actively recognize and bind E-selectin which is highly expressed on activated endothelial cells, so as to competitively inhibit the binding of tumor cells to E-selectin. The molecular weight of the polyethylene glycol is from 1500 to 20000.

In conclusion, the targeting drug delivery system herein has the advantages of dual targeting mechanisms (passive and active targeting), dual tumor killing mechanisms (tumor neovascularization and adjacent tumor cells) and dual therapeutic effects (inhibiting tumor growth and tumor metastasis). It has a great potential in the application of hydrophobic antitumor drugs.

The synthesis process of the surface double modified human serum albumin as a tumor targeting nano drug carrier herein is as follows.

E-selectin peptide ligand and its conjugate with mPEG were synthesized by Fmoc strategy using 2-chlorotriphenylmethyl chloride resin through solid-phase synthesis. They were represented by 8CR-Resin and PEG-8CR-Resin before cleavage from the resin.

The peptide conjugated with mPEG 2000 was synthesized by the following steps:

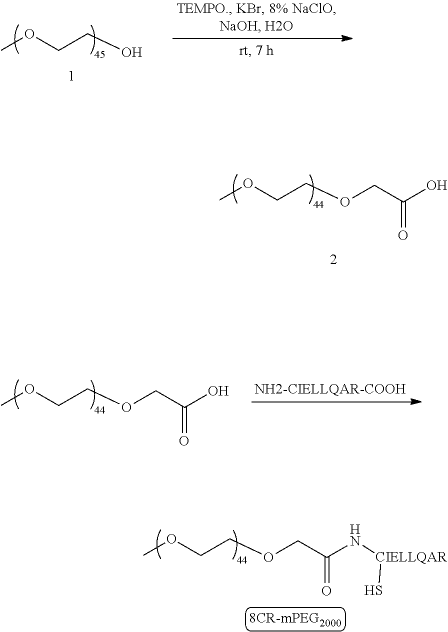

The conjugate (e.g., 8CR-mPEG2000) can react with activated HSA to prepare modified HSA monomer.

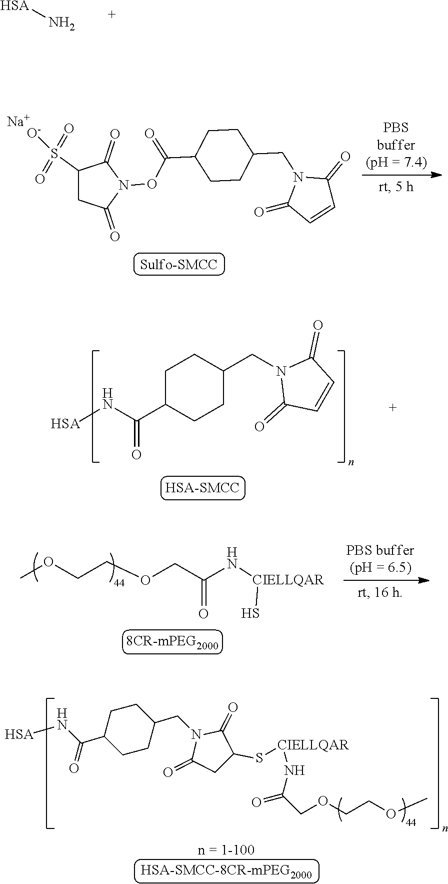

The above modified HSA monomer can form nanoparticles via intermolecular cross-linking with a diameter of 100-200 nm, at the same time, the drugs were loaded in the nanoparticles by hydrophobic interaction and other forces. Specifically, the modified HSA was dissolved in buffer solution, and β-mercaptoethanol was added to break intramolecular disulfide bond of HSA, then drugs (paclitaxel) were added. The free paclitaxel was removed by ultrafiltration and centrifugation, and the nanoparticles with drug loaded were obtained by freeze-drying. The mass ratio of HSA to drug can be from 1:1 to 20:1.

In order to explain the above synthetic method in detail, the following embodiments are provided.

### EXAMPLE 1

### Synthesis of Compound 2

2 g of polyethylene glycol monomethyl ether with an average molecular weight of 2000 Da was dissolved in 14 ml distilled water, and then 1 mg of 2,2,6,6-tetramethylpiperidine oxide and 0.2 g potassium bromide were added, stirred in ice bath, then 4.8 ml of 8% sodium hypochlorite solution was added, the pH was adjusted with 0.5N sodium hydroxide solution until the pH value was at about 10, then stirred at room temperature for 7 h, 0.7 mL ethanol was added for quenching reaction, and then HCl solution was added to adjust pH to 3. The mixture was extracted with dichloromethane, dried with anhydrous sodium sulfate, evaporated under reduced pressure, recrystallized with ether, and filtered to obtain 1.8 g white solid.

### EXAMPLE 2

### Synthesis of Compound 8CR-mPEG2000

0.674 g of 8CR-resin was added into 7 ml of DMF, 1.9 g of compound 2, 358 mg of HBTU, 127 mg of HOBt, 0.31 ml of 2-aminoethyldiisopropylamine were added and stirred for 2.5h at 30° C. The reaction was monitored by Ninhydrin until the resin was colorless. Then the resin was washed with 10 ml of DMF, isopropanol and DMF for 5 minutes in turn and then drained to obtain mPEG2000-8CR-Resin.

Preparation of solution for cleavage from the resin:

Phenol:ethyldithiol:Benzyl sulfide:water:trifluoroacetic acid=5:5:2.5:5:82.5 (V/V)

0.68 g mPEG2000-8CR-Resin was add into 6 ml cleavage solution in ice bath, and react at 30° C. for 2 h. Filter the reaction solution into cold ether and refrigerate overnight. After centrifugation, the obtained solid was washed with ether for three times, and then dried in argon to obtain 50 mg white powder 8CR-mPEG2000.

### EXAMPLE 3

### Synthesis of HSA-SMCC

3.125 mg of Sulfo-SMCC was add in 0.25 ml distilled water, stirred at about 40° C. to dissolve it, then 1.125 ml PBS (pH=7.4) buffer solution was added, the solution was then added to 8.94 ml PBS buffer solution containing 20 mg human serum albumin, stirred at room temperature for 5 h under argon. Dialyzed with a dialysis bag with cutoff weight of 8000-14000 Da in distilled water for 24 h, and then lyophilized to obtain 20 mg HSA-SMCC as white solid.

### EXAMPLE 4

### Synthesis of HSA-SMCC-8CR-mPEG2000

10 mg of HSA-SMCC was dissolved in 0.6 ml PBS (pH=6.5) buffer solution, and 6.3 mg 8CR-mPEG2000 was dissolved in 0.3 ml PBS (pH=6.5) buffer solution. The 8CR-mPEG2000 solution was slowly dropped into HSA-SMCC solution, stirred at room temperature for 16 h under argon, then dialyzed with a dialysis bag with cutoff weight of 3500 for 24 h, refrigerated and lyophilized to obtain 6 mg HSA-SMCC-8CR-mPEG2000 as white solid.

### EXAMPLE 5

### Synthesis of Targeting Nanoparticles

10 mg of HSA-SMCC-8CR-mPEG2000 was dissolved in 5 ml water, and 35 μL β-mercaptoethanol was added and stirred for 1 h to break the intramolecular disulfide bond of HSA. Add 2 mg paclitaxel (dissolved in 0.2 ml ethanol) and stir for 1 h. The insoluble paclitaxel was removed by centrifugation at 2000 r/min for 20 min, then the soluble free paclitaxel was removed by centrifugation. Finally, 15 mg paclitaxel loaded nanoparticles were obtained by freeze-drying. After freeze-drying, the nanoparticles can be easily redissolved with saline with similar size and zeta potential, the average particle size is 158 nm, the potential is −24.7 mv. The entrapment efficiency was detected up to 95% by HPLC.

### EXAMPLE 6

### Anti-Adhesion Activity of HSA-SMCC-8CR-mPEG2000

The present inventors chose HUVEC and HL-60 cells as anti-adhesion evaluation system, DMEMF-12 and 1640 media were used respectively. 1% penicillin-streptomycin was contained in the culture medium, 10% fetal bovine serum. The culture conditions are 37° C. and 5% CO₂ in constant temperature incubator.
(a) HUVEC cells were transplanted into 96 well black cell culture plate with a cell concentration of 1×10⁵ cells/ml. Three replicates were set for each experiment;
(b) After 36 h, the endothelial cells were activated (incubated at 37° C.) in fresh medium supplemented with TNF-α (20 ng/ml) for 6 h;
(c) Transfer HL-60 to 1.5 ml EP tube, 200 μl 1640 medium and 2 μl calcein-AM (final concentration 10 μm) were added, incubated for 45 min at 37° C.; after centrifugation, washed with PBS, cells were adjusted to 1×10⁶ cells/100 μL in 1640 medium for standby; and the compounds were diluted for standby too;
(d) After HUVEC cells were incubated for 6 h, the medium was discarded and the fluorescent labeled HL-60 cells were added, the compounds to be evaluated (final concentration was 250 μg/ml) were added immediately.
(e) After incubated in dark at 37° C., compounds competitively bind with E-selectin expressed on HUVEC surface with HL60 cells for 45 min;
(f) Washed with PBS buffer three times, and the fluorescence value (λₑₓ=485 nm, λₑₘ=528 nm) was measured by synergy 4 multifunctional microplate detector (BioTek).

The results showed that HSA-SMCC-8CR-mPEG2000 showed lower fluorescence intensity than that of the positive control group, indicating its anti-adhesion activity.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A surface double modified human serum albumin as targeting nano drug carrier characterized in that it includes human serum albumin, polyethylene glycol and E-selectin peptide ligand, wherein the E-selectin peptide ligand and polyethylene glycol are coupled to the surface of HSA.

2. The surface double modified human serum albumin as targeting nano drug carrier according to claim 1, wherein the sequence of E-selectin peptide ligand can be CIELLQAR, its reverse sequence analog thereof, or a peptide analog comprising an amino acid in a non-naturally occurring configuration (D-configuration).

3. The surface double modified human serum albumin as targeting nano drug carrier according to claim 1 characterized in that it comprises a hydrophobic antitumor drug loaded on the targeting nano drug carrier, wherein the hydrophobic antitumor drug includes one or more drugs selected from paclitaxel, adriamycin, docetaxel, etc.

4. The surface double modified human serum albumin as targeting nano drug carrier according to claim 1, characterized in that the molecular weight of the polyethylene glycol is from 1500 to 20000 Da.

5. A synthetic method of surface double modified human serum albumin as targeting nano drug carrier according to claim 1, characterized in the following steps:
(1) synthesizing a conjugate of E-selectin ligand and mPEG by Fmoc solid phase synthesis.
(2) coupling the conjugate of E-selectin ligand and mPEG with activated human serum albumin to obtain HSA monomer carrier.

6. The synthetic method according to claim 5, characterized in that the method in step (1) is as follows:

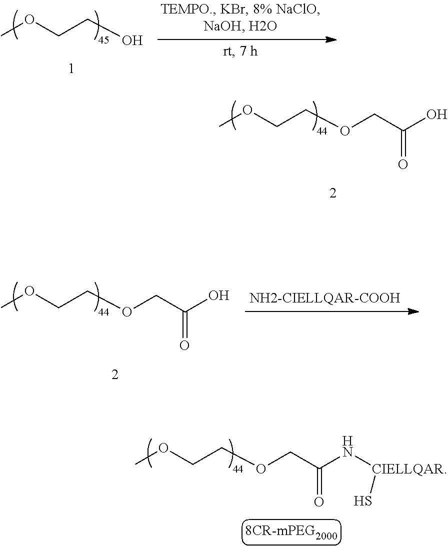

7. The synthetic method according to claim 5, characterized in that the method in step (2) is as follows:

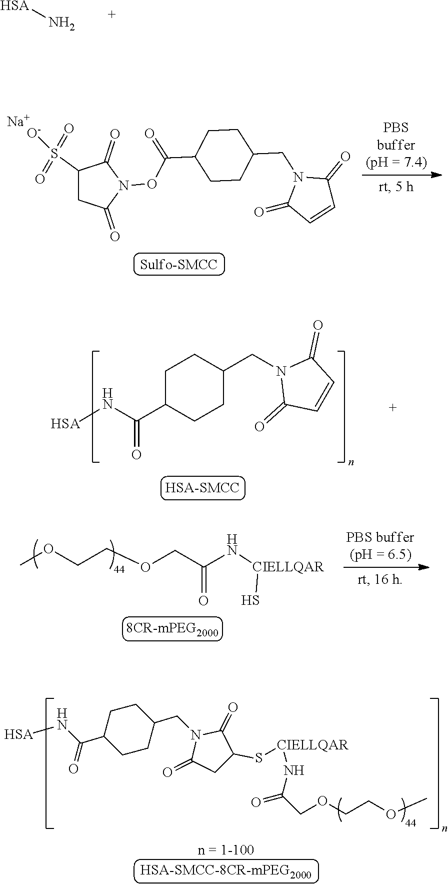

8. A drug loading method of the surface double modified human serum albumin as targeting nano drug carrier according to claim 1, characterized in that: dissolving the modified HSA in a buffer solution, adding β-mercaptoethanol to break the intramolecular disulfide bond, adding the drug to be loaded, removing the free drug by ultrafiltration and centrifugation, and freeze-drying to obtain the drug loaded nanoparticles.

9. Use of the targeting nano drug carrier according to claim 1 in the delivery of hydrophobic antitumor drugs.
